(19) United States
(12) Patent Application Publication
(10) Pub. No.: US 2024/0287445 A1
(43) Pub. Date: Aug. 29, 2024

(54) STREPTOMYCES ANTIBIOTICUS, PREPARATION OF METABOLITE THEREOF, AND USE THEREOF IN ANTIBACTERIAL ASPECT

(71) Applicants: PLANT PROTECTION RESEARCH INSTITUTE OF GUANGDONG ACADEMY OF AGRICULTURAL SCIENCES, Guangzhou, Guangdong (CN); GUANGDONG BRANCH OF CHINA TOBACCO GENERAL CO., LTD., Guangzhou, Guangdong (CN)

(72) Inventors: Qiyun YANG, Guangzhou, Guangdong (CN); Birun LIN, Guangzhou, Guangdong (CN); Zheng LU, Guangzhou, Guangdong (CN); Jingxin ZHANG, Guangzhou, Guangdong (CN); Dayuan SUN, Guangzhou, Guangdong (CN); Yonghua LV, Guangzhou, Guangdong (CN); Huifang SHEN, Guangzhou, Guangdong (CN); Xiaoming PU, Guangzhou, Guangdong (CN)

(21) Appl. No.: 18/549,090

(22) PCT Filed: Oct. 13, 2020
(86) PCT No.: PCT/CN2020/120625

§ 371 Date: Sep. 5, 2023

(30) Foreign Application Priority Data
May 7, 2020 (CN) 202010376108.6

(51) Int. Cl. C12N 1/20; A01N 43/24; A01N 63/28; A01P 3/00; C12N 3/00; C12P 17/08; C12R 1/48

(52) U.S. Cl. CPC C12N 1/20; A01N 43/24; A01N 63/28; A01P 3/00; C12N 3/00; C12P 17/08; C12R 2001/48

(57) ABSTRACT

A Streptomyces antibioticus PPI-16 with the accession number of GDMCC NO: 60970. Active compounds produced thereby can inhibit multiple pathogenic fungi, including Colletrichum orbiculare, Colletotrichum capsica, Tobacco anthracnose, and Candida albicans.

### INCORPORATION-BY-REFERENCE OF SEQUENCE LISTING

This application includes an electronically submitted sequence listing in .txt format. The .txt file contains a sequence listing entitled 2024-05-07-Sequence-Listing.txt created on May 7, 2024 and is 2,712 bytes in size. The sequence listing contained in this .txt file is part of the specification and is hereby incorporated by reference herein in its entirety.

### TECHNICAL FIELD

The present invention belongs to the field of biopesticide research, and particularly relates to a strain of Streptomyces antibiotics and a preparation of its metabolite and an application thereof in the aspect of disease resistance.

### BACKGROUND TECHNIQUE

With the widespread application of broad-spectrum antibiotics and immunosuppressive agents and the development of invasive treatment methods, new fungal diseases continue to appear, and the incidence of some ancient fungal diseases begins to rise, especially opportunistic fungal infections increase day by day. The incidence of fungal infections (especially invasive fungal infections) is increasing, causing serious harm to human health. The fungal infections happened to human being can be divided into three types: superficial fungal disease, deep fungal disease and systemic fungal disease (Garber, 2001), among which the most serious and life-threatening fungal infection is systemic fungal disease (Meis, 2001). The pathogens of the deep mycosis are mainly Candida, especially Candida albicans. It has the strongest pathogenicity among all species of Candida. Once the immunity of the body decreases or the normal balance of flora is destroyed, it will proliferate in abundance under mucosa and invade deep tissues, causing systemic candida infection (Maertens et al., 2001). Among nosocomial fungal infections, candida infection takes the first place, resulting in a fatality rate up to 21%. Candida can almost invade all organs of the body, and various candidas are still the most common source of fungal infection during hematopoietic stem cell transplantation (Zhou Qi et al., 2000). Although the body is sensitized by the opportunistic pathogen Candida albicans throughout its whole life, it has not developed to immune tolerance. Therefore, the incidence of Candida infection is on the rise, and the incidence of Candida ranks the first among immunosuppressed patients (Radentz, 1989). Candida albicans infections have increased by more than 40 times in the past 20 years, and have become one of the main causes for death in nosocomial infections (Liao Wanqing, 1985). Therefore, it is of great significance to search for new biological agentias of Candida.

Pepper and cucumber anthracnoses are common and serious fungal diseases in production, which can cause rotten leaves, rotten fruit and plant death. At present, chemical agentias such as carbendazim and mancozeb are mainly used in production for prevention and treatment, but the negative effects of pesticide residues, environmental pollution and the like are also gradually increasing. Therefore, it is necessary to find new biological agentias for the prevention and treatment of anthracnose.

### SUMMARY OF THE INVENTION

A primary purpose of the present invention is to overcome the shortcomings and deficiencies of the prior art and provide a strain of Streptomyces antibiotics.

Another purpose of the present invention is to provide a method for preparing the metabolite of the above-described Streptomyces antibiotics.

Another purpose of the present invention is to provide applications of the above-mentioned Streptomyces antibiotics and its metabolite.

The purpose of the present invention is achieved through the following technical solutions:

A strain of Streptomyces antibiotics, named Streptomyces antibioticus PPI-16, with the accession number of deposit of GDMCC NO: 60970, being deposited in Guangdong Microbial Culture Collection Center at 5th Floor, Building 59, Courtyard 100 Xianlie Middle Road, Guangzhou city on Mar. 5, 2020.

A spore of the Streptomyces antibiotics.

In the method for preparing the spore of the Streptomyces antibiotics, the Streptomyces antibiotics is inoculated on a culture medium and incubated at a condition of 28-30° C. to obtain the spore of the Streptomyces antibiotics.

The composition of the culture medium is: a soluble starch 1-5 g, a glucose 2-10 g, a yeast extract 1-3 g, a peptone 1-2 g, KNO₃ 0.5-1 g, K₂HPO₄ 0.1-0.5 g, NaCl 0.1-0.5 g, FeSO₄ 0.005-0.01 g, MgSO₄·7H₂O 0.01-0.05 g, and an agar 10-15 g, having a pH of 7.2-7.4, with water added to a constant volume to 1000 mL.

The soluble starch is preferably soluble corn starch.

The incubating time is 3 to 5 days.

A metabolite of Streptomyces antibiotics, is obtained by incubating the Streptomyces antibiotics and/or the spore of the Streptomyces antibiotics.

The preparation method for the metabolite of the Streptomyces antibiotics, includes the following steps:
(1) inoculating the Streptomyces antibiotics on a slant culture medium, and incubating the same at a condition of 28-30° C.;
(2) inoculating a strain on the slant culture medium into a fermentation culture medium for fermentation culture, removing a thallus by filtration to obtain a fermentation liquor; then concentrating the fermentation liquor to obtain a concentrate; extracting the concentrate with ethyl acetate and performing another concentrating to obtain a fermentation concentrate;
(3) subjecting the fermentation concentrate to chromatographic separation using a silica gel column, then washing with ethyl acetate and petroleum ether in a volume ratio of 3:20, then eluting with ethyl acetate and petroleum ether in a volume ratio of 1:1, and collecting the eluate (brown oily matter) to obtain the metabolite of the Streptomyces antibiotics.

The slant culture medium in the step (1) is: a soluble starch 1-5 g, a glucose 2-10 g, a yeast extract 1-3 g, a peptone 1-2 g, KNO₃ 0.5-1 g, K₂HPO₄ 0.1-0.5 g, NaCl 0.1-0.5 g, FeSO₄ 0.005-0.01 g, MgSO₄·7H₂O 0.01-0.05 g, and an agar 10-15 g, having a pH of 7.2-7.4, with water added to a constant volume to 1000 mL.

The soluble starch is preferably soluble corn starch.

The incubating time in the step (1) is 3 to 5 days.

The fermentation culture medium in the step (2) is: KNO₃ 0.5-1 g, K₂HPO₄ 0.1-0.5 g, MgSO₄·7H₂O 0.1-0.5 g, FeSO₄ 0.005-0.01 g, a seawater crystal 0.1-0.5 g, a soluble starch 2-10 g, a glucose 5-10 g, a yeast extract 15-20 g, constant volume to 1000 mL, having a pH of 7.2-7.4.

The soluble starch is preferably soluble corn starch.

The conditions for the fermentation culture in the step (2): performing the fermentation culture at room temperature for 5-7 days; preferably performing the fermentation culture at room temperature for 5 days.

The room temperature is preferably 28-30° C.; more preferably 28° C.

The concentrating in the step (2) is preferably concentrating by means of heating.

The concentrating in the step (2) is preferably concentrating to 1/20 to ⅓ of the volume of the fermentation liquor.

The another concentrating in the step (2) is preferably concentrating to ⅓ to ⅕ of the original volume.

The volume ratio of ethyl acetate to the concentrate in the step (2) is 1:1.

The silica gel in the silica gel column in the step (3) is 200-300 mesh silica gel.

The method for preparing the metabolite of the Streptomyces antibiotics, after the step (3), further includes the following steps:
separating the metabolite of the Streptomyces antibiotics obtained in the step (3) by high performance liquid chromatography to obtain an antimycin A1, wherein a mobile phase of the high performance liquid chromatography is a mixed solution of methanol and formic acid aqueous solution having a volume percentage of 0.5% in a volume ratio of 4:1.

An application of at least one of the Streptomyces antibiotics, the spore of the Streptomyces antibiotics, and the metabolite of the Streptomyces antibiotics in the production of the antimycin A1 (AntimycinA1).

An application of at least one of the Streptomyces antibiotics, the spore of the Streptomyces antibiotics, and the metabolite of the Streptomyces antibiotics in the prevention and treatment of fungal infections.

The fungus is plant anthracnose pathogens or Candida albicans.

The plant anthracnose pathogens include cucumber anthracnose pathogens, capsicum anthracnose pathogens and Tobacco anthracnose pathogens.

Compared with the prior art, the present invention has the following advantages and benefits:

The present invention screens and obtains a new strain (Streptomyces antibiotics PPI-16), which can produce a new active compound and can inhibit growth and reproduction of multiple pathogenic fungi, such as cucumber anthracnose pathogens, capsicum anthracnose pathogens, Tobacco anthracnose pathogens and Candida albicans, provide new resources for the development of new antibiotics. In addition, the present invention also provides the fermentation technology of an active substance produced by fermenting the Streptomyces antibiotics PPI-16.

### BRIEF DESCRIPTION OF THE FIGURES

FIG. 1 is a photograph of the spores of the Streptomyces antibiotics PPI-16.

FIG. 2 is a photograph of the spore hyphae of the Streptomyces antibiotics PPI-16.

FIG. 3 is a gel electrophoresis diagram of PCR products of the Streptomyces antibiotics PPI-16 16S rDNA (M: Marker; Lanes 1 and 2:16S rDNA of the Streptomyces antibiotics PPI-16).

FIG. 4 is a crystal diagram of the active ingredient of Streptomyces antibiotics PPI-16.

### DETAILED DESCRIPTION

The present invention will be further described in detail below in conjunction with examples, but the embodiments of the present invention are not limited thereto. Unless otherwise specified, the reagents, methods and equipment used in the present invention are conventional reagents, methods and equipment in the present technical field. The test methods without specific experimental conditions in the following examples are usually in accordance with the conventional experimental conditions or in accordance with the experimental conditions recommended by the manufacturer.

Unless otherwise specified, the reagents and raw materials used in the present invention are all commercially available.

### Example 1: Separation and Culture of Marine Actinomycetes

1. Biological materials and culture medium: the biological materials used to separate marine actinomycetes were collected from mangrove forests in Enping City, Guangdong Province, a total of 792 samples. The culture medium used for the separation of actinomycetes is Gao's No. 1 culture medium (in order to ensure the number and types of the separated actinomycetes, the separation culture medium is prepared with artificial seawater, and the formula is: a soluble corn starch 20 g, KNO₃ 1 g; K₂HPO₄ 0.5 g; MgSO₄·7H₂O 0.5 g; FeSO₄·7H₂O 0.01 g; NaCl 0.5 g; agar 20 g; and artificial seawater 1000 mL, pH 7.2-7.4; wherein the formula of the artificial seawater is: NaCl 24.477 g; MgCl₂·6H₂O 4.981 g; Na₂SO₄ 3.917 g; CaCl₂) 2H₂O 1.102 g; KCl 0.664 g; NaHCO₃0.192 g; KBr 0.096 g; H₃BO₃ 0.026 g, SrCl₂ 0.024 g; NaF 0.0039 g; and distilled water 1000 mL). In the following experiments, the culture media used for routinely incubating actinomycetes were all prepared with distilled water.

2. Sample processing: the above-mentioned freshly collected biological sample was putted in a foam box with an ice bag, and brought back to the laboratory for processing as soon as possible. After the sample is weighed, it is washed with sterile sea water for 3 to 5 times to remove the impurities attached to the surface; the washed sample was placed in a sterile mortar, and sterile artificial sea water was added according to 1:8 (W/V) (The formula is the same as the above step 1) for homogenization. 1 mL of the homogenate solution was sucked in a centrifuge tube and heated in a water bath at 55° C. for 6 minutes; the sample after the warm bath was sucked and diluted in a gradient of multiples of 10, and 100 μL of each dilution was taken and applied to the above-described separation culture medium. In the base, each treatment is repeated 3 times.

3. Actinomycetes culture: the separation culture medium coated with the homogenate solution was placed in a constant temperature incubator at 28° C. and incubated insertedly. After 7 days of incubation, a single colony grown on the culture medium was picked for transfer purification. According to the morphological characteristics and culture characteristics of the strains, the repeated strains were removed, and a total of 792 different actinomycetes strains were finally separated. The purified strains were inoculated into Gao's No. 1 slant culture medium, cultured in a constant temperature incubator at 28° C., and stored in a refrigerator at 4° C. for later use.

### Example 2: Screening, Separation and Identification of Antagonistic Marine Actinomycetes

### 1. Screening and Separation of Antagonistic Marine Actinomycetes

1.1 Strains and culture media for test: Candida albican (purchased from the Guangdong Institute of Microbiology) was used as an indicator bacterium to determine the inhibitory activity of 792 strains of the separated actinomycetes against Candida albicans, to screen out the strains with the strongest inhibitory activity against Candida. The culture medium used for the culture of actinomycetes is Gao's No. 1 culture medium (the specific formula is as described in Example 1); the culture medium used for the culture of Candida albicans is Martin culture medium (the formula is: glucose 20 g; peptone 5 g; Yeast extract 4 g; K₂HPO₄·7H₂O 0.63 g; MgSO₄·7H₂O 1.8 g; and agar 15 g; with water added to make the volume to 1000 mL).

1.2 Activation of the strain: the corresponding culture medium was prepared according to the above formula, the culture medium in an Erlenmeyer flask was heated and dissolved, and poured into a petri dish with a diameter of 9 cm to prepare a culture medium plate. The actinomycetes stored in the refrigerator at 4° C. were respectively picked and inoculated on the Gao's No. 1 culture medium plate, and placed in a constant temperature incubator at 28° C. for 3 to 5 days.

### 1.3 Screening of Strains

1.3.1 Preliminary screening: the paper diffusion method (Kirby Bauer, KB method) (Hunfeld, 2001) was used to determine the inhibitory activity of the sample against Candida. The Martin solid culture medium was poured to the plate, and 0.1 mL (with a concentration of 1×10⁶ cfu/ml) Candida albicans solution was inoculated, and coated evenly. A sterile tweezer was used to affix the filter paper (with a diameter of 6 mm) containing the actinomycete liquor to the surface of the plate evenly. After incubating at 37° C. for 1 day, the diameter of the inhibition zone was measured and repeated for three times. The preliminary screening results were shown as Table 1 (names of the strain in the table are all self-named).

TABLE 1
Results of preliminary screening of active
strains against Candida albicans.

| | Inhibition | | Inhibition | | Inhibition |
|---|---|---|---|---|---|
| Name | zone | Name | zone | Name | zone |
| of | diameter | of | diameter | of | diameter |
| Strain | (mm) | Strain | (mm) | Strain | (mm) |
| H122-01 | 25 | H122-02 | 25 | H113-10 | 20 |
| H113-06 | 15 | H112-05 | 8 | H91-14 | 10 |
| PPI-16 | 21 | H74-21 | 20 | H74-5 | 11 |
| H121-09 | 12 | H121-03 | 9 | H20-39 | 8 |
| H87-7 | 15 | H83-3 | 14 | H112-01 | 25 |
| H13-13 | 12 | H20-38 | 8 | H121-04 | 9 |
| H55-20 | 11 | H55-24 | 15 | H55-36 | 15 |
| H73-18 | 9 | H75-11 | 24 | H72-4 | 16 |
| H72-12 | 11 | H66-2 | 15 | H122-03 | 25 |
| H129-06 | 10 | H105-04 | 11 | H117-03 | 30 |
| H127-06 | 11 | H82-11 | 10 | H106-12 | 12 |
| H112-04 | 9 | H96-4 | 10 | H106-01 | 15 |
| H100-4 | 11 | H109-1 | 11 | H97-7 | 14 |
| H118-04 | 25 | H116-06 | 25 | H128-01 | 25 |
| 99-01 | 6 | 133-12 | 7 | 114-04 | 7 |
| 200-09 | 17 | 113-02 | 7 | 111-11 | 15 |
| 92-04 | 15 | 200-17 | 8 | 108-01 | 9 |
| 21-01 | 9 | 135-04 | 9 | 28-04 | 10 |
| 115-09 | 10 | 96-21 | 10 | 89-07 | 10 |
| 95-3 | 9 | 177-08 | 15 | 95-05 | 8 |
| 155-19 | 11 | 162-02 | 11 | 172-07 | 11 |
| 140-03 | 11 | 170-02 | 11 | 90-03 | 11 |
| 108-02 | 12 | 177-13 | 12 | 101-04 | 13 |
| 191-09 | 14 | 13¹ | 14 | 195-07 | 14 |
| 196-05 | 20 | 45-06 | 15 | 200-12 | 16 |

1.3.2 Re-screening: strains of actinomycetes with good initial screening effect (with a diameter of inhibition zone for Candida >15 mm) (seeing Table 1) were selected, and the strains were rejuvenated and incubated on the Gao's No. 1 solid culture medium. After incubating for 2 generations, a sterile punch was used to take out a 6 mm in diameter colony. It was placed in Martin's culture medium coated with Candida albicans. After incubating at 37° C. for 1 day, the diameter of the inhibition zone was measured and the measurement was repeated for three times.

Re-screening is to select strains with an inhibition zone diameter of 15 mm or more, wherein the strain PPI-16 has the best antibacterial effect against Candida albicans (seeing Table 2), and the inhibition zone diameter is 20.3 mm.

TABLE 2
Results of re-screening of active strains against Candida albicans

| | Inhibition zone | | Inhibition zone |
|---|---|---|---|
| Name of Strain | diameter (mm) | Name of Strain | diameter (mm) |
| H122-01 | 19.3 ± 0.12 b | H75-11 | 15.1 ± 0.12 f |
| H74-21 | 16.4 ± 0.09 d | H122-02 | 15.9 ± 0.09 e |
| 200-09 | 17.5 ± 0.21 c | PPI-16 | 20.3 ± 0.13 a |

Note:
The data in the table is the average of 3 replicates ± SE, and the analysis of variance is performed by DPS. Those with the same letter indicate that the difference is not significant at the 5% level.

Through the above method, a strain PPI-16 with stronger antibacterial ability against Candida albicans was re-screened, from the 6 separated strains (strains with an inhibitory zone diameter of 15 mm or more for Candida) actinomycetes.

### 2. Identification of the Strain PPI-16

### 2.1 Morphological Observation

The morphological characteristic of the strain was observed using the plate insert method (Yan Xunchu, 1992). The test strains were inoculated on 7 kinds of solid culture medium, including synthetic starch, Krebs, Chase, glucose aspartin agar, potato pieces and Gao's No. 1, slices were inserted and cultured at 28° C. for 7-20 days. The growth and the color of the intrabasal hyphae, aerial hyphae and soluble pigments were observed and recorded. The inserts were taken out at the same time, and observation was performed on the aerial hyphae, the morphology of the basic hyphae, and whether there were diaphragms, breaks, swelling and the like characteristics with an optical microscope. After the selected cultures were fixed, dehydrated, etc., they were observed and photographed by a scanning electron microscope.

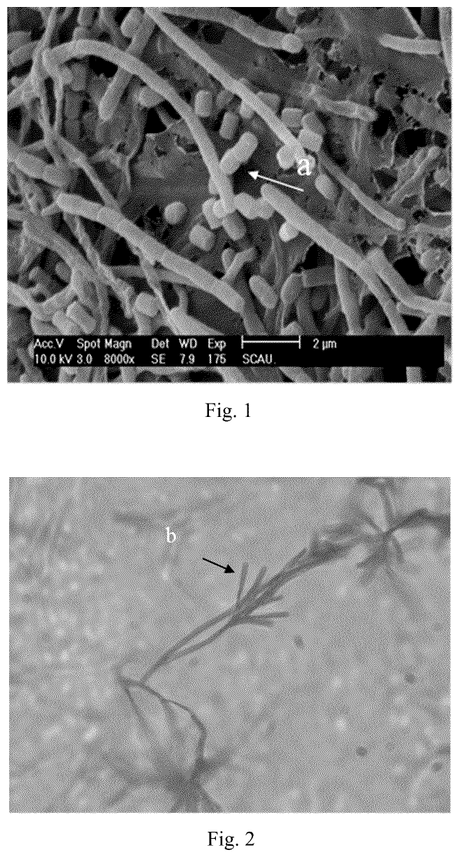

The results were shown as FIGS. 1 and 2: FIG. 1 was the spores of the Streptomyces antibiotics PPI-16, and FIG. 2 was the spore hyphae of the Streptomyces antibiotics PPI-16. After the strain was incubating on Gao's No. 1 agar for 7-10 days, the basal hyphae and aerial hyphae were abundant, the basal filaments were not broken, the spore hyphae were grown on the aerial hyphae, the spore hyphae were flexible, and the spores were smooth and cylindrical. The number of spores on the mature spore chain was about 8, and no cysts, nuclei and the like structures were observed.

### 2.2 Physiological and Biochemical Characteristics

The strain PPI-16 was identified according to the conventional identification method of Streptomyces, specifically: determining liquefaction of gelatin, hydrolysis of starch, coagulation and peptonization of milk, hydrolysis of cellulose of the strain, Carbon source utilization and the like characteristics with reference to the method of Lechevalie (Lechevalier et al., 1980), while using Streptomyces antibioticus (EF063450) and Streptomyces griseoruber (AB184209) (both deriving from Guangdong Institute of Microbiology) as controls.

The results are shown as Table 3: The results show that PPI-16 could liquefy gelatin, could hydrolyze starch, did not grow on cellulose, did not peptonize or liquefy milk, did not produce melanin, and could utilize 6 types of carbon sources.

TABLE 3
Comparison of carbon source utilization between PPI-16
and Streptomyces antibiotics and Streptomyces griseus

| | Streptomyces | | Streptomyces |
|---|---|---|---|
| Characteristic | antibiotics | PPI-16 | griseus |
| Glucose | + | + | + |
| Mannitol | + | + | − |
| L-rhamnose monohydrate | + | + | + |
| Sucrose | − | + | − |
| Raffinose | − | + | − |
| Inositol | + | + | + |
| L-Arabinose | + | − | + |
| Fructose | + | − | + |
| Xylose | + | − | + |

Note:
+ means that the carbon source can be used, − means that the carbon source cannot be used.

### 2.3 Analysis for Chemical Composition of Cell Wall

Microcrystalline cellulose thin-layer chromatography was used for whole-cell amino acid and whole-cell hydrolysate glycoform analysis. Whole-cell hydrolysate of the strain PPI-16 contained L,L-DAP (L−) (diaminopimelic acid), glycine, alanine and aspartic acid, contained ribose and glucose, and had no characteristic sugars (glycotype C). The cell wall component belonged to type I, which accorded with the characteristics of the chemical classification of Streptomyces.

2.4 Determination and Analysis for 16S rDNA Sequence

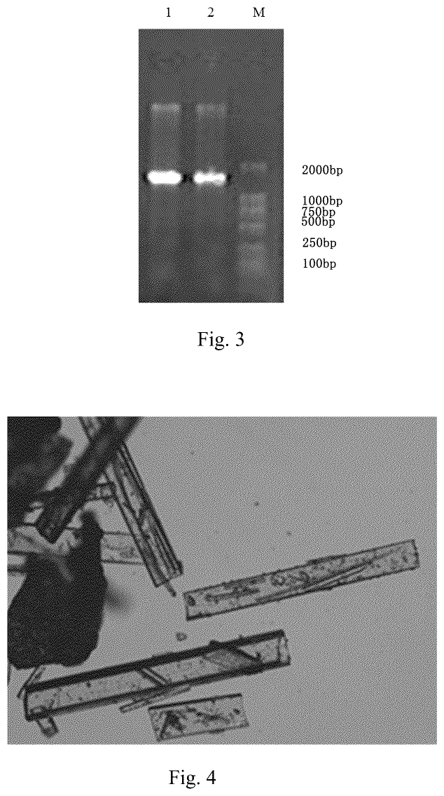

Using the forward primer PA (5′-AGAGTTTGATCATGGCTCAG-3′) and the reverse primer PB (5′-AAGGAGGTGATCCAGCCGCA-3′) as primers, the 16S rDNA sequence of the strain PPI-16 was obtained by PCR amplification, and the electrophoresis results were shown as FIG. 3. The following results were obtained upon measuring: it sequence length is 1440 bp and the content of G+C is 59%.

ATGCAAGTCGAACGATGAAGCCCTTCGGGGTGGATTAGTGGCGAACGGG
TGAGTAACACGTGGGCAATCTGCCCTGCACTCTGGGACAAGCCCTGGAA
ACGGGGTCTAATACCGGATATCACTCTTGCAGGCATCTGTGAGGGTCGA
AAGCTCCGGCGGTGCAGGATGAGCCCGCGGCCTATCAGCTTGTTGGTGA
GGTAATGGCTCACCAAGGCGACGACGGGTAGCCGGCCTGAGAGGGCGAC
CGGCCACACTGGGACTGAGACACGGCCCAGACTCCTACGGGAGGCAGCA
GTGGGGAATATTGCACAATGGGCGAAAGCCTGATGCAGCGACGCCGCGT
GAGGGATGACGGCCTTCGGGTTGTAAACCTCTTTCAGCAGGGAAGAAGC
GAAAGTGACGGTACCTGCAGAAGAAGCGCCGGCTAACTACGTGCCAGCA
GCCGCGGTAATACGTAGGGCGCAAGCGTTGTCCGGAATTATTGGGCGTA
AAGAGCTCGTAGGCGGCTTGTCACGTCGGGTGTGAAAGCCCGGGGCTTA
ACCCCGGGTCTGCATTCGATACGGGCTAGCTAGAGTGTGGTAGGGGAGA
TCGGAATTCCTGGTGTAGCGGTGAAATGCGCAGATATCAGGAGGAACAC
CGGTGGCGAAGGCGGATCTCTGGGCCATTACTGACGCTGAGGAGCGAAA
GCGTGGGGAGCGAACAGGATTAGATACCCTGGTAGTCCACGCCGTAAAC
GGTGGGAACTAGGTGTTGGCGACATTCCACGTCGTCGGTGCCGCAGCTA
ACGCATTTAAGTTCCCCGCCTGGGGAGTACGGCCGCAAGGCTAAAACTC
AAAGGAATTGACGGGGGCCCGCACAAGCAGCGGAGCATGTGGCTTAATT
CGACGCAACGCGAAGAACCTTACCAAGGCTTGACATACACCGGGAAACG
GCCAGAGATGGTCGCCCCCTTGTGGTYGGTGTACAGGTGGTGCATGGCT
GTCGTCAGCTCGTGTCGTGAGATGTTGGGTTAAGTCCCGCAACGAGCGC
AACCCTTGTTCTGTGTTGCCAGCATGTCCTTCGGGATGATGGGGACTCA
CAGGAGACCGCCGGGGTCAACTCGGAGGAAGGTGGGGACGACGTCAAGT
CATCATGCCCCTTATGTCTTGGGCTGCACACGTGCTACAATGGCCGGTA
CAAAGAGCAGCGATACCGTGAGGTGGAGCGAATCTCAAAAAGCCGGTCT
CAGTTCGGATTGGGGTCTGCAACTCGACCCCATGAAGTCGGAGTTGCTA
GTAATCGCAGATCAGCATTGCTGCGGTGAATACGTTCCCGGGCCTTGTA
CACACCGCCCGTCACGTCACGAAAGTCGGTAACACCCGAAGCCGGTGGC
CCAACCCCTTGTGGGAGGGAGCTGTCGAAGGTGGGACTGGCGATTGGGA
CGAAGTCGTAACAAGGTAA.

An analysis for BLAST similarity between the sequence and the related data in GenBank was performed, 84 similar sequences were searched, and 9 typical strain sequences that are more similar to the 16S rDNA sequence of PPI-16 were selected for comparison. These strains were shown as Table 4:

TABLE 4
Strains similar to PPI-16 in GenBank

| Subject ID | Strain | Identity % |
|---|---|---|
| EF063450 | S. antibioticus | 99 |
| AB184209 | S. griseoruber | 99 |
| AB184534 | S. spinichromogenes | 98 |
| AB184266 | S. cinnabarinus | 98 |
| AB184192 | S. cellostaticus | 98 |
| AB045860 | S. panayensis | 98 |
| AB184461 | S. roseogriseus | 98 |
| AB184536 | S. viridochromogenes | 98 |
| AB184387 | S. griseochromogenes | 98 |

According to the classification and identification manual of actinomycetes and streptomyces, morphological characteristics, culture characteristics (short and straight spore hyphae, long cylindrical and smooth-surface spores) and physiological and biochemical characteristics of the strain PPI-16 exhibited the typical characteristics of Streptomyces, belonging to the golden group. A phylogenetic tree was constructed along with the 16S rDNA of the 9 strains of Streptomyces with the highest homology rate. The strain PPI-16 and S. antibioticus EF063450 were in the same branch and had the closest genetic relationship, wherein the homology was as high as 99.90. However, cultivated characteristics, physiological and biochemical characteristics thereof were quite different from those of Streptomyces antibioticus, so PPI-16 was identified as a new species of Streptomyces antialbonostocticus of Streptomyces aureus.

TABLE 5
Comparison of Streptomyces antialbonostocticus with Streptomyces
antibioticus and Streptomyces griseoruber

| Characteristic | S. antibioticus | S. antialbonostocticus | S. griseoruber |
|---|---|---|---|
| Spore hypha | Long and straight | Short and straight | Loose, tight spiral |
| Spore | Spherical to slightly oval, | Long cylindrical, smooth | Oval and short cylinder, |
| | smooth | | smooth |
| Liquefaction of | Slow at the beginning of | Fast liquefaction, no | Slow liquefaction, brown |
| Gelatin | liquefaction, and fast at the | pigment | to light yellow-green |
| | later phase of liquefaction, | | |
| | melanin | | |
| growth of Cellulose | Weak growth | Does not grow | Does not grow |
| Peptonization of Milk | Peptonized but not solidified | neither peptonize nor | Solidify and peptonize |
| | | solidify | |
| The production of | Produce | Not producing | Produce |
| H2S | | | |
| Pigment (starch agar) | black | yellow | None or yellowish brown |

The present invention named the strain PPI-16 obtained by screening as Streptomyces antibioticus PPI-16, and this strain was deposited in Guangdong Microbial Culture Collection Center at 5th Floor, Building 59, Courtyard 100 Xianlie Middle Road, Guangzhou city on Mar. 5, 2020, with the accession number of deposit being GDMCC NO: 60970.

### Example 3 Preparation of Metabolite

An extract of the present invention can be obtained by extraction and separation from the ethyl acetate extract of the culture (fermentation liquor) of marine actinomycetes PPI-16. The specific preparation method thereof includes the following steps:

(1) Seed Incubation of Marine Actinomycetes PPI-16:

The strains were selected and inoculated to the slant culture medium and incubated at 28-30° C. for 3 to 5 days; wherein,

The culture medium is (by weight ratio): soluble corn starch 5 g; glucose 10 g; yeast extract 3 g; peptone 2 g; KNO₃ 1 g; K₂HPO₄ 0.5 g; NaCl 0.5 g; FeSO₄ 0.01 g; MgSO₄·7H₂O 0.05 g; and agar 15 g, having a pH of 7.4; and diluted to a constant volume of 1000 mL with water; and a test tube slant was made.

(2) Fermentation Incubating of Marine Actinomycetes PPI-16:

The strains incubated in the slant (with an inoculation amount of 5%) were picked into the fermentation culture medium, and fermented and incubated at room temperature 28° C. for 5 days; wherein, the fermentation culture medium (by weight ratio): KNO₃ 1 g, K₂HPO₄ 0.5 g, MgSO₄·7H₂O 0.5 g, FeSO₄ 0.01 g, seawater crystal 0.5 g, soluble corn starch 10 g, glucose 10 g, and yeast extract 20 g, diluted to a constant volume of 1000 mL with water, and having a pH of 7.4.

(3) Filtering the Above-Mentioned Incubated Fermentation Liquor to Remove the Thallus;

(4) The fermentation liquor was heated and concentrated to 1/20 of the volume of the original liquor, was then extracted with ethyl acetate having equal volume for 3 times, and the ethyl acetate extract liquor was concentrated (to ⅓ of the original volume), to obtain 15 g of condensed substance. Chromatographic separation was carried out in a silica gel column (200-300 mesh), and it was washed with ethyl acetate/petroleum ether in a ratio of 15% (volume percentage) and then eluted with ethyl acetate/petroleum ether in a ratio of 50% (volume percentage) as an eluent, at a flow rate of 10 ml/min; and

(5) 50% (volume percentage) of the ethyl acetate/petroleum ether eluate was collected and concentrated to obtain a brown oily matter (5 g), which was the extract (metabolite) required by the present invention. The effective ingredient crystals observed under the microscope from Zeiss were shown as FIG. 4.

### Example 4: Prevention and Control Test of the Metabolite of the Present Invention on Anthracnose Pathogen of Cucumber and Pepper

### (1) Preparation of Test Sample Solution

1 mg of the extract of the present invention (red-brown oily matter prepared in Example 3) was taken and ultrasonically dissolved in 1.0 mL of 10% (v/v) methanol solution.

### (2) Preparation of Spore Suspension of Anthracnose Pathogen of Cucumber and Pepper

A potato dextrose agar (PDA) culture medium (the preparation method is: weighing 200 g potato, washing, peeling and chopping, adding water to 1000 mL, boiling for half an hour, filtering with gauze, adding 20 g glucose and 20 g agar to fully dissolve, filtering with gauze while being hot, dispensing into glass test tubes, and sterilizing at 121° C. and a high pressure for 20 min) was used for incubating Colletrichum orbiculare (Colletorichum cucumisativus) and anthracnose pathogen of pepper (Colletotrichum capsici) at 28° C., and the hyphae was scraped with sterilized distilled water, to respectively prepare a spore suspension (concentration of 1×10⁶ cfu/ml).

### (3) Bacteriostatic Test

5 μL of the test sample solution was pipetted and added on a sterilized filter paper (6 mm), and the same was placed on a solid agar plate coated with indicator bacteria (cucumber anthracnose pathogen and capsicum anthracnose pathogen), and incubated in a constant temperature incubator for 48 hours. Thereafter, the results were observed and the antibacterial activity was judged according to the size of the inhibition zone. The results are as follows:

TABLE 6
Antibacterial activity of PPI-16 mycelial extract

| pathogenic bacteria for test | Inhibition zone diameter(mm) |
|---|---|
| Colletorichum cucumisativus | 21.1 ± 0.51a |
| Colletotrichum capsici | 15.5 ± 0.25d |

Note:
The data in the table is the average of 3 replicates ± SE, and the analysis of variance is performed by DPS. Those with the same letter indicate that the difference is not significant at the 5% level.

### Example 5 Inhibition Experiment of the Metabolite of the Present Invention on Candida albicans

The paper diffusion method (Kirby Bauer, KB method) (Hunfeld, 2001) was used to determine the inhibitory activity of the sample against Candida. The specific steps are as follows:

(1)

### Preparation of Test Sample Solution

1 mg of the extract of the present invention (red-brown oily matter prepared in Example 2) was taken and ultrasonically dissolved in 1.0 mL of 10% (v/v) methanol solution.

### (2) Bacteriostatic Test

Martin solid culture medium (the formula is: glucose 20 g, peptone 5 g, yeast extract 4 g, K₂HPO₄·7H₂O 0.63 g, MgSO₄·7H₂O 1.8 g, agar 15 g, and water 1000 mL) was prepared, and poured into the petri dish. 0.1 mL Candida albicans (with a concentration of 1×10⁶ cfu/ml) (Candida albicans strain is purchased from Guangdong Institute of Microbiology) culture solution was inoculated, and coated evenly. Then, sterile tweezers was used to flatly stick the filter paper (diameter 6 mm) containing the test sample solution (5 μL) on the surface of the solid plate, and clean water was used as the control. After incubating at 37° C. for 1 day, the diameter of the inhibition zone against Candida was measured.

TABLE 7
Anti-Candida activity of extracts

| Extract | Inhibition zone diameter(mm) |
|---|---|
| PPI-16 | 21.3 ± 0.10a |
| Control | 0 |

Note:
The data in the table is the average of 3 replicates ± SE, and the analysis of variance is performed by DPS. Those with the same letter indicate that the difference is not significant at the 5% level.

### Example 6

The crystal obtained in Example 3 was analyzed as mixtures by DionexP680 analytical high performance liquid chromatographic instrument (product of American Dionex Co., with a detector of PDA-100 Photodiode Array Detector). Compound A was obtained by separating and preparing via a preparative high performance liquid chromatography (methanol/0.5% formic acid aqueous solution, in a volume ratio of 80:20), and subjected to a LC-MS in combination (AB 4000Q Traq liquid chromatography-mass spectrometer from American Agilent Co.). Chromatographic analysis conditions were: Agilent Eclipse Plus C 18 (5 μm) chromatographic column (2.1 mmx 150 mm); mobile phase: A (100.0% methanol): B (0.5% (V/V) formic acid aqueous solution)=80:20; flow speed: 300 μL/min; detection wavelength: 240 nm. Its chemical structure was technically analyzed. The results showed that compound A was Antimycin A1 and its chemical structure was as follows:

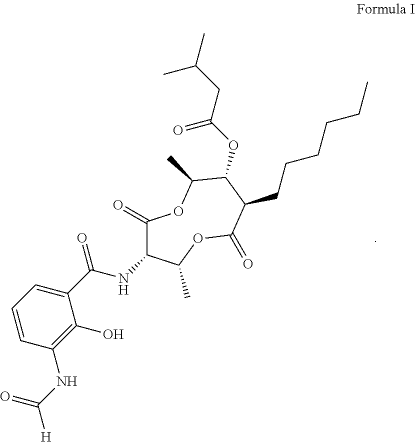 [SMILES: CCCCCC[C@H]1C(=O)O[C@H](C)[C@H](NC(=O)c2cccc(NC=O)c2O)C(=O)O[C@@H](C)[C@@H]1OC(=O)CC(C)C]

The above-mentioned examples are preferred embodiments of the present invention, but the embodiments of the present invention are not limited by the above-mentioned examples, and any other changes, modifications, substitutions, combinations and simplifications made without departing from the spirit and principle of the present invention, should all be equivalent replacement modes, and they are all included in the protection scope of the present invention.

1. A strain of Streptomyces antibioticus, including a name of Streptomyces antibioticus PPI-16, and an accession number of deposit of GDMCC NO: 60970, and wherein this strain is deposited in Guangdong Microbial Culture Collection Center at 5th Floor, Building 59, Courtyard 100 Xianlie Middle Road, Guangzhou city on Mar. 5, 2020.

2. A spore of the Streptomyces antibioticus according to claim 1.

3. A preparation method for a spore of the Streptomyces antibioticus according to claim 1, wherein inoculating the Streptomyces antibioticus on a culture medium, and incubating it at a condition of 28-30° C. to obtain the spore of the Streptomyces antibioticus;
components of the culture medium are: a soluble starch 1-5 g, a glucose 2-10 g, a yeast extract 1-3 g, a peptone 1-2 g, KNO₃ 0.5-1 g, K₂HPO₄ 0.1-0.5 g, NaCl 0.1-0.5 g, FeSO₄ 0.005-0.01 g, MgSO₄·7H₂O 0.01-0.05 g, and an agar 10-15 g, having a pH of 7.2-7.4, with water added to a constant volume to 1000 mL; and
the incubating time is 3-5 days.

4. A metabolite of a Streptomyces antibioticus, obtained by incubating the Streptomyces antibioticus according to claim 1 and/or a spore of the Streptomyces antibioticus.

5. A preparation method for the metabolite of the Streptomyces antibioticus according to claim 4, comprising the following steps:
(1) inoculating the Streptomyces antibioticus on a slant culture medium, and incubating it at a condition of 28-30° C.;
(2) inoculating the strain on the slant culture medium into a fermentation culture medium for fermentation culture, removing a thallus by filtration to obtain a fermentation liquor; then concentrating the fermentation liquor to obtain a concentrate; extracting the concentrate with ethyl acetate and then performing another concentrating to obtain a fermentation concentrate; and
(3) subjecting the fermentation concentrate to chromatographic separation by a silica gel column, then washing with ethyl acetate and petroleum ether in a volume ratio of 3:20, then eluting with ethyl acetate and petroleum ether in a volume ratio of 1:1, and collecting the eluate to obtain the metabolite of the Streptomyces antibioticus.

6. The preparation method for the metabolite of the Streptomyces antibioticus according to claim 5, wherein
the slant culture medium in the step (1) is: a soluble starch 1-5 g, a glucose 2-10 g, a yeast extract 1-3 g, a peptone 1-2 g, KNO₃ 0.5-1 g, K₂HPO₄ 0.1-0.5 g, NaCl 0.1-0.5 g, FeSO₄ 0.005-0.01 g, MgSO₄·7H₂O 0.01-0.05 g, and an agar 10-15 g, having a pH of 7.2-7.4, with water added to a constant volume to 1000 mL;
the incubating time in the step (1) is 3-5 days;
the fermentation culture medium in the step (2) is: KNO₃ 0.5-1 g, K₂HPO₄ 0.1-0.5 g, MgSO₄·7H₂O 0.1-0.5 g, FeSO₄ 0.005-0.01 g, a seawater crystal 0.1-0.5 g, a soluble starch 2-10 g, a glucose 5-10 g, and a yeast extract 15-20 g, with a constant volume to 1000 mL, having a pH of 7.2-7.4;
the fermentation culture condition in the step (2) is: performing the fermentation culture at room temperature for 5-7 days;
the concentrating in the step (2) is concentrating by means of heating;
the concentrating in the step (2) is concentrating to 1/20-3/1 of the volume of the fermentation liquor;
the another concentrating in the step (2) is concentrating to ⅓-⅕ of the original volume;
the volume ratio of the ethyl acetate to the concentrate in the step (2) is 1:1; and
the silica gel in the silica gel column in the step (3) is a 200-300 mesh silica gel.

7. The preparation method for the metabolite of the Streptomyces antibioticus according to claim 5, comprising the following steps after the step (3):
separating the metabolite of the Streptomyces antibioticus obtained in the step (3) by high performance liquid chromatography to obtain an antimycin A1; wherein a mobile phase of the high performance liquid chromatography is a mixed solution of methanol with a formic acid aqueous solution having a volume percentage of 0.5% in a volume ratio of 4:1.

8. An application of at least one of the Streptomyces antibioticus according to claim 1, a spore of the Streptomyces antibioticus, and a metabolite of the Streptomyces antibioticus obtained by incubating the Streptomyces antibioticus and/or the spore of the Streptomyces antibioticus in the production of an antimycin A1, wherein the antimycin A1 has a chemical structural formula shown as Formula I

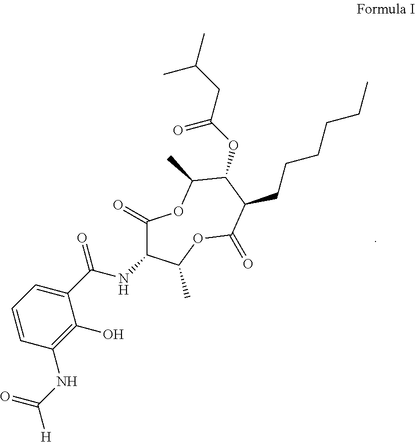 [SMILES: CCCCCC[C@H]1C(=O)O[C@H](C)[C@H](NC(=O)c2cccc(NC=O)c2O)C(=O)O[C@@H](C)[C@@H]1OC(=O)CC(C)C]

9. An application of at least one of the Streptomyces antibioticus according to claim 1, a spore of the Streptomyces antibioticus, and a metabolite of the Streptomyces antibioticus obtained by incubating the Streptomyces antibioticus and/or the spore of the Streptomyces antibioticus in the prevention and treatment of a fungal infection,
wherein the fungus is plant anthracnose pathogens or Candida albicans; and the plant anthracnose pathogens includes Colletotrichum orbiculare, Colletotrichum capsica and Tobacco anthracnose.

10. (canceled)


SEQUENCE LISTING (3 sequences)

SEQ ID NO: 1 - DNA, 20 bp, Artificial Sequence
  Forward primer PA
agagtttgatcatggctcag

SEQ ID NO: 2 - DNA, 20 bp, Artificial Sequence
  Reverse primer PB
aaggaggtgatccagccgca

SEQ ID NO: 3 - DNA, 1440 bp, Streptomyces antibioticus
atgcaagtcgaacgatgaagcccttcggggtggattagtggcgaacgggtgagtaacacgtgggcaatctgccctgcactctgggacaagccctggaaacggggtctaataccggatatcactcttgcaggcatctgtgagggtcgaaagctccggcggtgcaggatgagcccgcggcctatcagcttgttggtgaggtaatggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcgggtgtgaaagcccggggcttaaccccgggtctgcattcgatacgggctagctagagtgtggtaggggagatcggaattcctggtgtagcggtgaaatgcgcagatatcaggaggaacaccggtggcgaaggcggatctctgggccattactgacgctgaggagcgaaagcgtggggagcgaacaggattagataccctggtagtccacgccgtaaacggtgggaactaggtgttggcgacattccacgtcgtcggtgccgcagctaacgcatttaagttccccgcctggggagtacggccgcaaggctaaaactcaaaggaattgacgggggcccgcacaagcagcggagcatgtggcttaattcgacgcaacgcgaagaaccttaccaaggcttgacatacaccgggaaacggccagagatggtcgcccccttgtggtyggtgtacaggtggtgcatggctgtcgtcagctcgtgtcgtgagatgttgggttaagtcccgcaacgagcgcaacccttgttctgtgttgccagcatgtccttcgggatgatggggactcacaggagaccgccggggtcaactcggaggaaggtggggacgacgtcaagtcatcatgccccttatgtcttgggctgcacacgtgctacaatggccggtacaaagagcagcgataccgtgaggtggagcgaatctcaaaaagccggtctcagttcggattggggtctgcaactcgaccccatgaagtcggagttgctagtaatcgcagatcagcattgctgcggtgaatacgttcccgggccttgtacacaccgcccgtcacgtcacgaaagtcggtaacacccgaagccggtggcccaaccccttgtgggagggagctgtcgaaggtgggactggcgattgggacgaagtcgtaacaaggtaa